(19) United States
(12) Patent Application Publication
(10) Pub. No.: US 2023/0200841 A1
(43) Pub. Date: Jun. 29, 2023

(54) OSTOMY WAFER CUTTER

(71) Applicants: Steven J. Wright, SeaTac, WA (US); Krista J. Wright, SeaTac, WA (US)

(72) Inventors: Steven J. Wright, SeaTac, WA (US); Krista J. Wright, SeaTac, WA (US)

(21) Appl. No.: 17/561,810

(22) Filed: Dec. 24, 2021

(51) Int. Cl. A61B 17/3205; A61F 5/443; A61F 5/445; B26F 1/38; B26F 1/44

(52) U.S. Cl. CPC A61B 17/32053; A61F 5/443; A61F 5/445; B26F 1/44; B26F 1/3846; B26F 2001/4472

(57) ABSTRACT

Provided is an ostomy wafer cutter for enlarging a hole of an ostomy wafer. The ostomy wafer cutter includes an upper element having a locking means to removably lock a blade element. An upper arm is connected to the upper element. A lower element comprises a platform and a central pin for insertion in the hole of the ostomy wafer. A lower arm is connected to the lower element. A handle portion positioned on at least one of: upper arm and/or lower arm, the handle portion graspable and moveable by a user in a cutting position to bring the upper element in close contact with the lower element causing the cutting edge to cut the ostomy wafer enlarging the hole of the ostomy wafer. The proposed wafer cutter efficiently enlarges the opening of the ostomy wafer and is useful for patients having limited dexterity and/or for elderly people.

### FIELD OF INVENTION

The present invention generally relates to a medical instrument. More particularly, the present invention relates to a cutter for enlarging a hole in the ostomy wafer for use by medical patients.

### BACKGROUND

Various surgical procedures such as colostomy, ileostomy, and urostomy and so on require creating of an artificial stoma. The waste contents of the body organs such as kidneys, abdomen, bladder and so on can be eliminated through the stoma instead of through rectum or anus. These surgical procedures require attaching of a barrier, also known as ostomy wafer, to the skin of the patients. Typically, the ostomy wafer is adhered to the skin around the stoma and it provides a means for maintaining an ostomy bag in fluid communication with the stoma. The ostomy bag accumulates the waste contents of body organs.

Generally, the ostomy wafer is fabricated from an elastic material which enables it to conform and adhere to the skin with minimal discomfort to the patient and prevents it from absorbing substantial amount of waste contents. The ostomy wafer has an adhesive backing layer for adhering to the body of the patient.

Typically, the ostomy wafer comprises a central opening which must correspond as closely as possible to the size and shape of the stoma. If the opening is too big, the skin of the patient is subjected to irritation and/or infection caused by exposure of waste contents. If the opening is too small, the opening can block the passage of waste contents through the stoma.

Generally, the standard precut opening in the ostomy wafer has a smaller circular shape and size such that the ostomy wafer could be utilized for various applications as stoma for various body organs can vary greatly. Thus, each patient must cut the central opening in the ostomy wafer to conform to the dimensions of the particular stoma and/or ostomy bag.

Generally, scissors are the most commonly used tool to make these cuts. The person using them has to make a cut through the ostomy wafer and around the proper size outline on the wafer for their needs which could leave jagged/rough edges on the surfaces of the circular opening which could cause irritation and/or infection around the stoma.

Currently, various hand cutters are used to enlarge the central opening of the ostomy wafer. However, the hand cutters can make an oblong cut if not positioned correctly on the ostomy wafer. The person using the hand cutter has to line up the cutter to the cut lines on the wafer, then push down on the hand cutter to create the circular cut. This is difficult and not precise for the elderly and people with limited dexterity. Thus, it could leave jagged/rough edges on the surfaces of the circular opening which could cause irritation and/or infection around the stoma.

Such as for example, US5079843A discloses an adjustable size hole cutter for ostomy adhesive wafers is disclosed having firstly a cylindrical body adapted to hold a cutting blade, a cylindrical pilot protruding outward from the bottom circular face of the cylindrical body to reside in a preformed hole in the colostomy wafer, and a Tee handle protruding outward from the top face. Adjustment of the blade’s distance from the center of the cylindrical body for cutting different sized holes is accomplished by means of removable adjustment spacers situated in a radial slot formed in the wall of the cylindrical body, the cutting blade secured to the outside adjustment spacer by a set screw passing through a hole in the blade and aligned holes in the adjustment spacers. A circular base plate with centrally located hole receives the pilot of the adjustable hole cutter. The ostomy wafer is placed between the bottom face of the cylindrical body and the base plate whereupon the blade cuts into the wafer and the circular hole cut as the cutter is rotated.

The conventional cutting methods and/or tools accomplish their purposes of enlarging the opening of the ostomy wafer, yet they do require a substantial amount of force from the patient which could be a problem for elderlypersons and/ or patients having limited dexterity. Further, the conventional cutting methods and/or tools does not effectively and efficiently enlarge the opening of the ostomy wafer as it could leave jagged/rough edges on the surfaces of the circular opening which could cause irritation and/or infection around the stoma.

In the light of foregoing, there is a need of an improved ostomy wafer cutter which effectively and efficiently enlarges the opening of the ostomy wafer as well as could be useful for those patients having limited dexterity and/or elderly people. Further, the improved ostomy wafer cutter should cause minimal chance to cause abrasions which can lead to infection of the stoma. Thus, an improved ostomy wafer cutter to address the aforementioned problems is desired.

### BRIEF SUMMARY

Before the present systems and methods, embodiments are described, it is to be understood that this application is not limited to the particular systems, and methodologies described, as there can be multiple possible embodiments which are not expressly illustrated in the present disclosures. It is also to be understood that the terminology used in the description is for the purpose of describing the particular versions or embodiments only, and is not intended to limit the scope of the present application.

It is an object of the present invention to provide an ostomy wafer cutter for enlarging a hole of an ostomy wafer comprising an upper element comprising a locking means to removably lock a blade element. The blade element comprises a cutting edge. An upper arm is connected to an upper element. A lower element comprises a platform, and a central pin is arranged in the platform for insertion in the hole of the wafer. A lower arm is connected to a lower element. A handle portion is positioned on atleast one of: upper arm and/or lower arm. A user may grasp and move the handle portion in a cutting position to bring the upper element in close contact with the lower element causing the cutting edge to cut the ostomy wafer and thereby enlarging a hole of the ostomy wafer.

In an embodiment, the lower arm is rotatably connected to the upper arm by a hinge element.

In an embodiment, the blade element comprises a biasing spring to restore the upper element relative to the lower element in the initial position.

In an embodiment, a spring is positioned between the upper arm and the lower arm to restore the upper arm relative to the lower arm in the initial position.

In an embodiment, the locking means comprises atleast one of: a twist lock arrangement, luer locks and screw arrangement.

In an embodiment, the platform has a sharp cutting edge.

In an embodiment, the platform comprises a circumferential slot.

In an embodiment, the handle portion comprises a scissors handle.

In an embodiment, the handle portion comprises a stapler handle.

Various objects, features, aspects and advantages of the present invention will become more apparent from the following detailed description of the embodiments of the invention, along with the accompanying drawings in which like numerals represent like components.

### BRIEF DESCRIPTION OF THE DRAWINGS

The foregoing summary, as well as the following detailed description of preferred embodiments, is better understood when read in conjunction with the appended drawings. There is shown in the drawings example embodiments, however, the application is not limited to the specific system and method disclosed in the drawings.

FIG. 1 illustrates a perspective view of an ostomy wafer cutter, according to an embodiment of the present invention;

FIG. 2 illustrates a bottom view of an upper element of the ostomy wafer cutter of FIG. 1, according to an embodiment of the present invention;

FIG. 3 illustrates a bottom view of a blade element of the ostomy wafer cutter of FIG. 1, according to an embodiment of the present invention;

FIG. 4 illustrates a side view of the blade element of FIG. 3.

FIG. 5 illustrates a cross sectional view of the blade element of FIG. 3.

FIG. 6 illustrates a cross sectional view of a blade element, according to another embodiment of the present invention; wherein the blade element comprises a biasing spring.

FIG. 7 illustrates an assembled position of the blade element and the upper element.

FIG. 8 illustrates cross sectional views of a set of cutting profiles of the blade element, according to various embodiments of the present invention;

FIG. 9 illustrates side views of another set of cutting shapes of the blade element, according to various other embodiments of the present invention;

FIG. 10 illustrates a top view of a platform of a lower element of the ostomy wafer cutter of FIG. 1, according to an embodiment of the present invention;

FIG. 11 illustrates a cross sectional view of the platform of FIG. 10.

FIG. 12 illustrates a perspective view of an ostomy wafer cutter, according to another embodiment of the present invention, wherein the platform has a cutting edge;

FIG. 13 illustrates a cross sectional view of the platform of FIG. 12.

FIG. 14 illustrates a perspective view of an ostomy wafer cutter, according to another embodiment of the present invention, wherein the platform has a circular relief portion.

FIG. 15 illustrates a top view of the platform of FIG. 14.

FIG. 16 illustrates a cross sectional view of the platform of FIG. 14.

FIG. 17 illustrates a perspective view of an ostomy wafer cutter, according to another embodiment of the present invention, wherein the handle portion has a scissor handle;

FIG. 18 illustrates a perspective view of an ostomy wafer cutter, according to another embodiment of the present invention; wherein the ostomy wafer cutter has a hinge-less design.

FIG. 19 illustrates a conventional prior art wafer having a hole.

### DETAILED DESCRIPTION

Some embodiments, illustrating its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Although any methods, and systems similar or equivalent to those described herein can be used in the practice or testing of embodiments, the preferred methods, and systems are now described. The disclosed embodiments are merely exemplary.

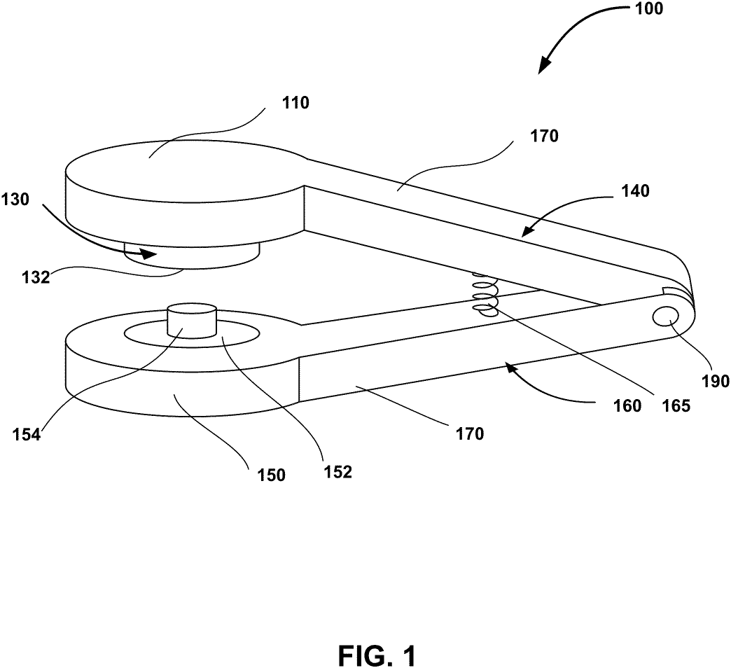

The various features and embodiments of an ostomy wafer cutter of the present invention will now be described in conjunction with the accompanying figures, namely FIGS. 1-19.

FIG. 1 depicts a perspective view of an ostomy wafer cutter 100, according to an embodiment of the present invention. The ostomy wafer cutter 100 comprises an upper element 110, an upper arm 140, a lower element 150 and a lower arm 160.

In an embodiment as shown in FIG. 1, the upper arm 140 is integrally connected to an upper element 110 and the lower arm 160 is integrally connected to the lower element 150 respectively. Further, the lower arm 160 is rotatably connected to the upper arm 140 by a hinge element 190. A handle portion 170 is positioned on both upper arm 140 and lower arm 160 for manipulating the position of upper arm 140 relative to the lower arm 160.

In another embodiment (not shown in figures), the upper arm 140 is detachably connected to an upper element 110 and the lower arm 160 is detachably connected to the lower element 150 respectively. This configuration enables a user to easily store the ostomy wafer cutter 100 by detaching parts of the ostomy wafer cutter 100. Further, this configuration enables easy replacement of damaged parts due to prolonged use. The upper arm 140 is detachably connected to an upper element 110 and the lower arm 160 is detachably connected to the lower element 150 respectively by a suitable means such as but not limited to: locking screws, friction couplings, keys and keyways, friction couplings, luer locks, mechanical locks, pins, cotter joints, magnetic couplings and so on.

In another embodiment (not shown in figures), the handle portion 170 comprises an ergonomically designed grip of a suitable material such as but not limited to: rubber, silicon, neoprene and so on to ensure easy grasping by the hands of the user. In another embodiment (not shown in figures), the handle portion 170 could be configured as a stapler handle.

In another embodiment (not shown in figures), the handle portion 170 is positioned on either one of the upper arm 140 and the lower arm 160.

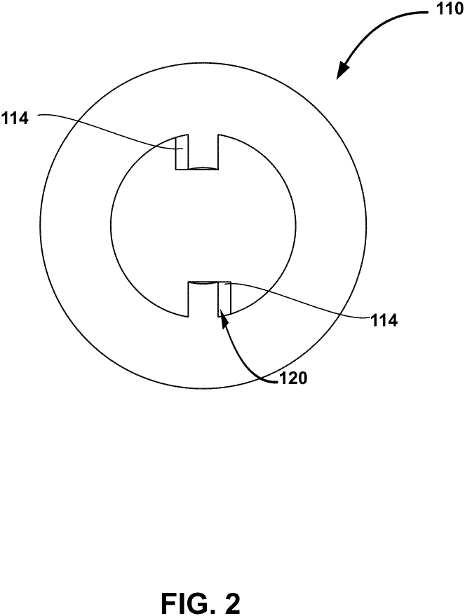

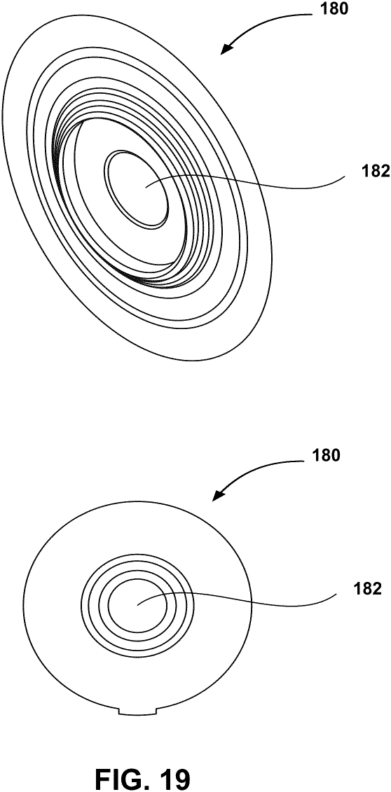

The upper element 110 comprises a locking means 120 (FIG. 2) to removably lock a blade element 130 which will be described in detail in the below description. The blade element 130 comprises a cutting edge 132. The lower element 150 comprises a platform 152. A central pin 154 is arranged in the platform 152 for insertion in the hole 182 (FIG. 19) of the ostomy wafer 180 (FIG. 19) which will be described in detail in the below description.

A user can manipulate the handle portion 170 using their hands in at least two positions: (a) a cutting position in which the upper element 110 is in close contact with the lower element 150, causing the cutting edge 132 to cut the ostomy wafer 180 (FIG. 19) and thereby enlarging a hole 182 (FIG. 19) of the ostomy wafer 180 (FIG. 19); and an initial position in which the upper element 110 is kept at a sufficient distance apart from the lower element 150.

An exemplary method of cutting an enlarged hole using the ostomy wafer cutter 100 will now be described with reference to FIGS. 1-19.

As shown in FIG. 19, a conventional prior art wafer 180 comprising a hole 182 is shown. The hole 182 is already precut by the manufacturer of the wafer 180. The standard precut hole 182 in the ostomy wafer 180 has a smaller circular shape and size such that the ostomy wafer could be utilized for various applications as stoma for various body organs can vary greatly.

Firstly, a patient places an ostomy wafer 180 (FIG. 19) on the lower element 150 such that the central pin 154 is inserted in a hole 182 (FIG. 19) of the ostomy wafer 180 (FIG. 19). Initially, the upper element 110 is at an initial position wherein the upper element 110 is at a sufficient distance from the lower element 150. Afterwards, the upper element 110 is brought in a cutting position by manipulating handle portion 170 such that the upper element 110 comes in close contact with the lower element, thereby, causing the cutting edge 132 of the blade element 130 to cut the ostomy wafer 180 and thereby enlarging a hole 182 of the ostomy wafer 180. Thus, a new enlarged hole 182′ (not shown in figures) is formed due to cutting action of the blade element 130. The platform 152 is formed of such as material such that it absorbs impact of the cutting edge 132 of the blade element 130 without damage (wear and tear) of the platform 152. Afterwards, a spring 165 positioned between the upper arm 140 and the lower arm 160 restores the upper arm 140 relative to the lower arm 160 back in the initial position due to biasing action of the spring 165.

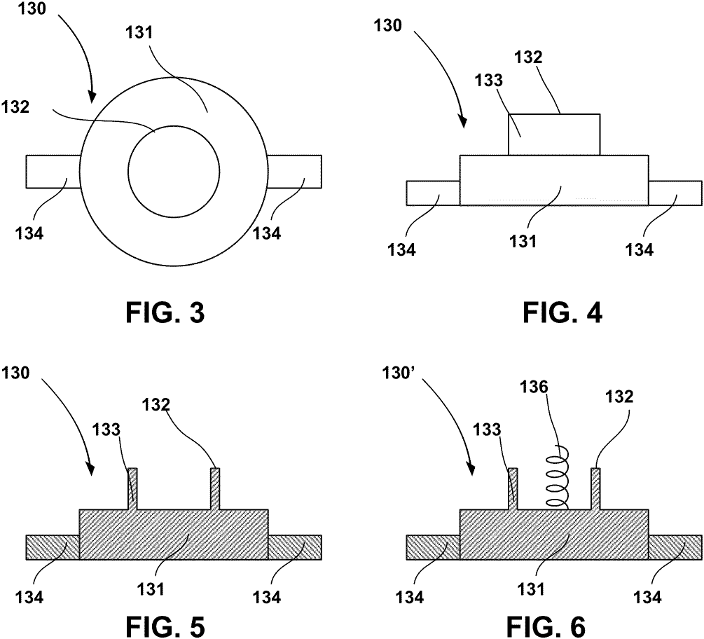

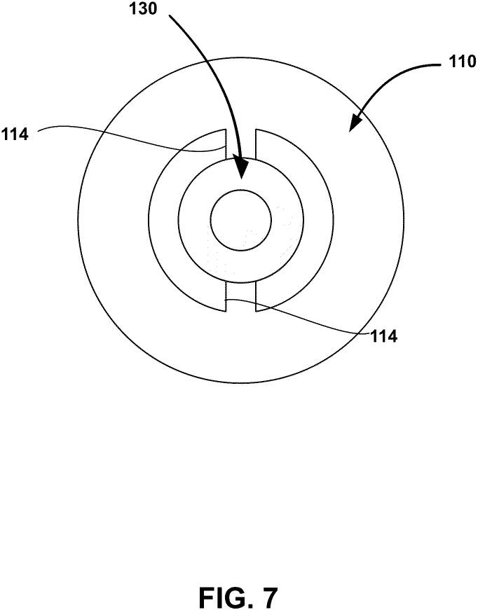

Referring to FIG. 2 and FIG. 3 depicting bottom views of the upper element 110 and the blade element 130 respectively, the upper element 110 comprises a locking means 120 to removably lock a blade element 130. As shown in FIG. 2, the locking means 120 comprises a twist lock mechanism. The twist lock mechanism comprises a pair of blade extensions 134 positioned on diametrically opposite edges of the blade element 130 and extending radially from the blade element 130. Further, the twist lock mechanism comprises a pair of one way holes 114 positioned on diametrically opposite edges in the upper element 110. The twist lock mechanism is configured such that the when the blade element 130 is rotated in a specific direction (clockwise direction), the blade extensions 134 are moved in the one way holes 114 such that each blade extension 134 is received in a one way hole 114, thereby bringing the blade element 130 in the assembled position as shown in FIG. 7. On the other hand, when the twist lock is rotated in an opposite direction (clockwise direction), the blade extensions 134 are moved out from the one way holes 114 such that each blade extension 134 is displaced from a one way hole 114, thereby bringing the blade element 130 in the disassembled position.

In another embodiment (not shown in figures), the locking means 120 could include means such as but not limited to : luer locks, screw arrangements, magnetic couplings, mechanical locks, friction locks, snap fit couplings, keys and keyways and so on.

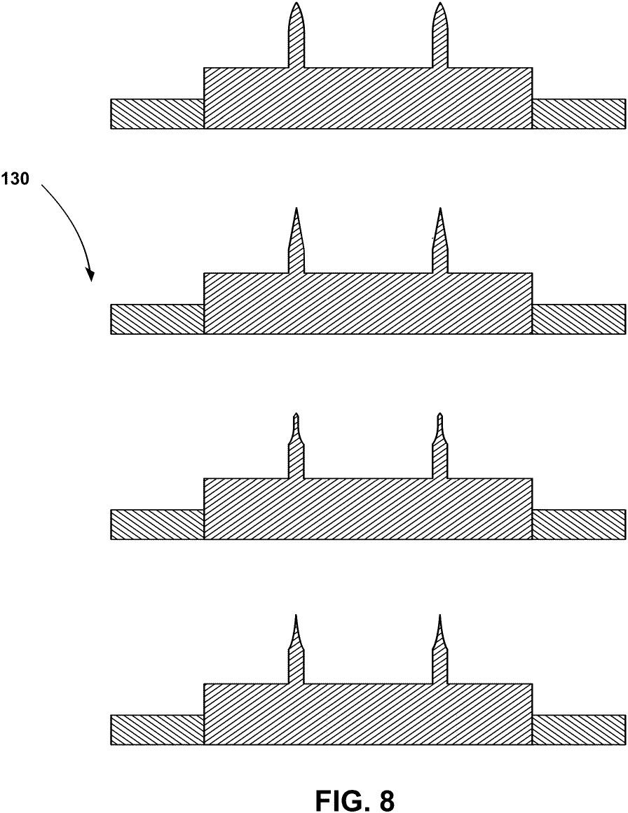

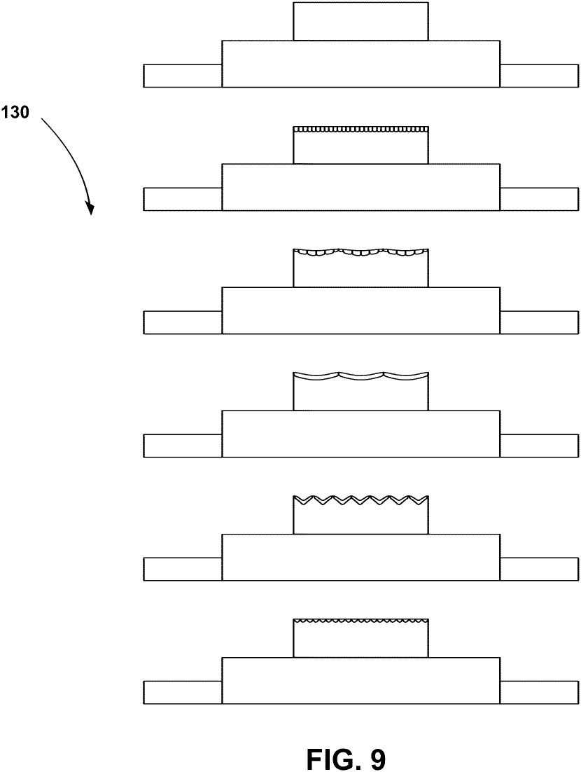

Referring to FIG. 4 and FIG. 5, the blade element 130 further comprises a base element 131 connected to a hollow cylindrical element 133. The hollow cylindrical element 133 has a cutting edge 132. In an embodiment as shown in FIG. 4, the cutting edge 132 has a substantially straight shape. In other embodiments, the cutting edge 132 could have other shapes such as but not limited to: multiple serrated shapes, which could be non-straight as seen in FIG. 9. In an embodiment as shown in FIG. 5, the cutting edge 132 has a substantially flat profile (flat tip). In other embodiments, the cutting edge 132 could have other profiles such as but not limited to: conical tip, convex tip, frustoconical tip, spheroconical tip as seen in FIG. 8.

Referring to FIG. 6, an embodiment of the blade element 130′ is shown. The blade element 130′ comprises a base element 131 connected to a hollow cylindrical element 133.The hollow cylindrical element 133 has a cutting edge 132. The blade element 130′ is similar to the blade element 130 shown in FIG. 5 except for modifications associated with the spring. The attachment clip 100′ comprises a biasing spring 136 to restore the upper element 110 relative to the lower element 150 in the initial position due to biasing action of spring 136. As shown in FIG. 6, the length of the biasing spring 136 is larger than the length of the hollow cylindrical element 133. In another embodiment (not shown in figures), the length of the biasing spring 136 could be kept smaller or equal to the length of the hollow cylindrical element 133.

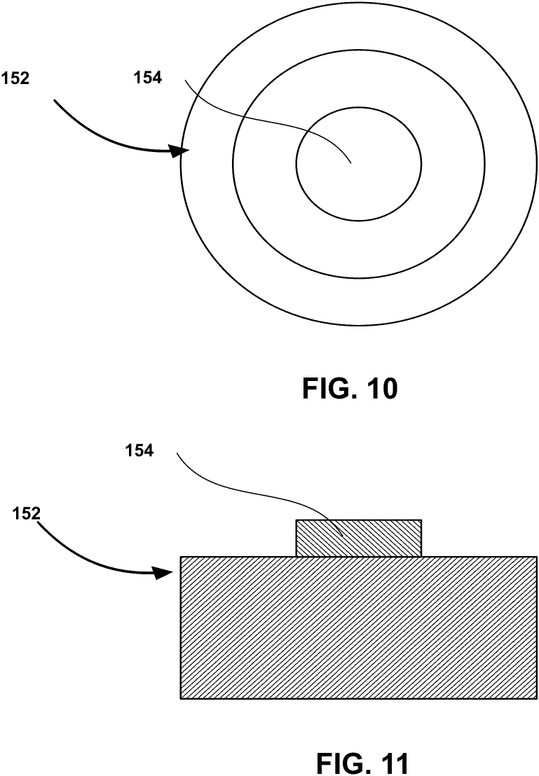

Referring to FIG. 10 and FIG. 11 depicting top view and cross sectional view of the platform 152 of the lower platform 150 respectively. The central pin 154 is arranged in the platform 152 for insertion in the hole 182 (FIG. 19) of the ostomy wafer 180 (FIG. 19). The overall diameter of the central pin 154 is substantially equal to the hole 182 (FIG. 19) of the ostomy wafer 180 (FIG. 19). The platform 152 is formed of such as material such that it absorbs impact of the cutting edge 132 of the blade element 130 without wear and tear of the platform 152. The material could include but not limited to: high density rubber, plastics, metals and so on.

Further, in an embodiment (not shown in figures), the platform 152 is replaceable to allow a new platform 152 to be used on the lower element 150 after removing a substantially damaged platform 152 due to prolonged use. The platform 152 is removably connected to the lower element 150 by a suitable means such as but not limited to: screws and threads, friction couplings, snap-fit couplings, pins , mechanical locks, magnetic couplings, luer locks , cotter joints and so on. Further, in an embodiment (not shown in figures), the lower element 150 could comprise a hole (not shown in figures) for insertion of the platform 152 in the hole (not shown in figures) of the lower element 150.

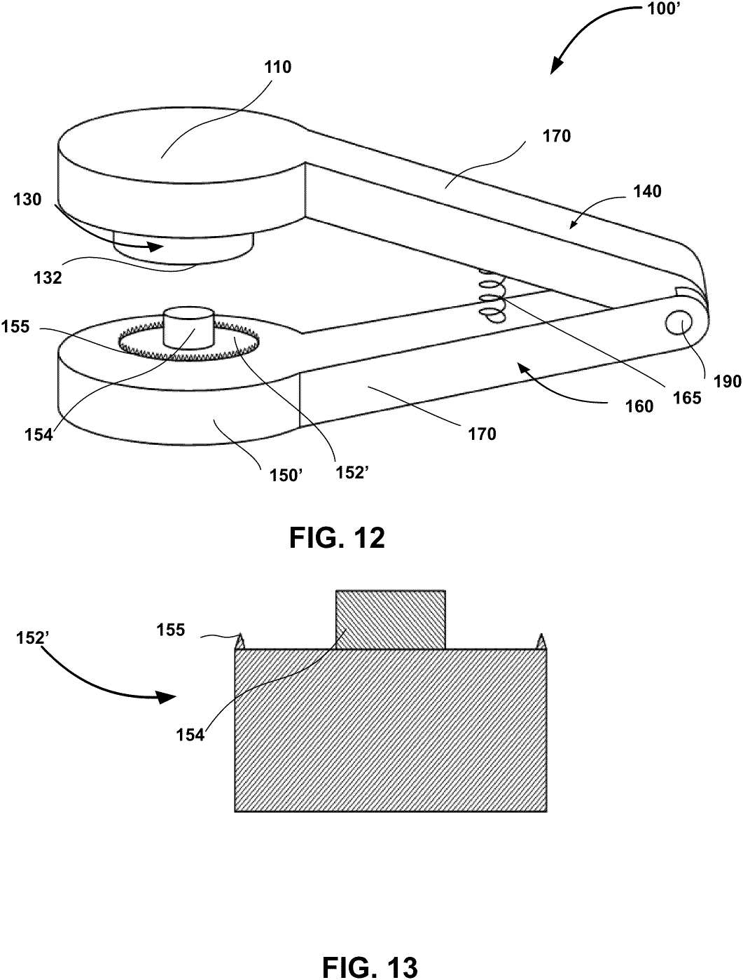

Referring to FIG. 12 that depicts a perspective view of an ostomy wafer cutter 100′, according to another embodiment of the present invention. The ostomy wafer cutter 100′ is similar to the ostomy wafer cutter 100 shown in FIG. 1 except for modifications associated with the platform of the lower element. As shown in FIG. 12 and FIG. 13, a platform 152′ of the lower element 150′ comprises a cutting edge 155 which further assists in cutting of the hole 182 (FIG. 19) of the ostomy wafer 180 (FIG. 19). The ostomy wafer 180 (FIG. 19) is sandwiched between the cutting edge 132 of the blade element 130 and the platform 152′. Thus, the cutting action of the hole 182 (FIG. 19) of the ostomy wafer 180 (FIG. 19) is performed simultaneously by the cutting edge 155 of the platform 152′ and the cutting edge 132 of the blade element 130.

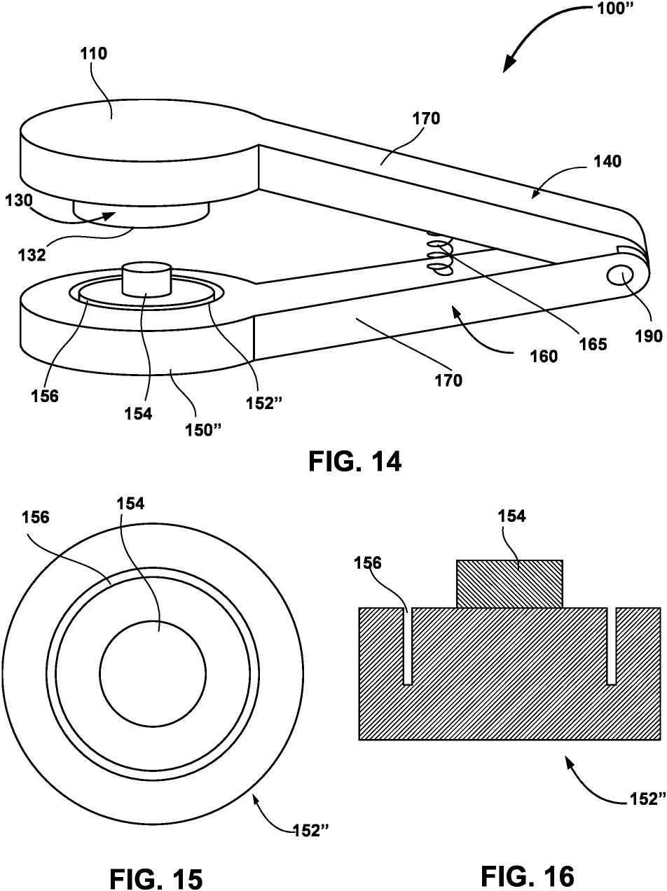

Referring to FIG. 14, that depicts a perspective view of an ostomy wafer cutter 100″ according to another embodiment of the present invention. The ostomy wafer cutter 100″ is similar to the ostomy wafer cutter 100 shown in FIG. 1 except for modifications associated with the lower element. As shown in FIG. 14, FIG. 15 and FIG. 16, the platform 152″ comprises a circumferential slot 156 formed in the platform 152″. The circumferential slot 156 is substantially circular in shape and the circumferential slot 156 is configured such that it allows the cutting edge 132 of the blade element 130 to pass below the top surface of the platform 152″ to cut the ostomy wafer 180 (FIG. 19) wherein the ostomy wafer 180 (FIG. 19) is placed on the platform 152″. This configuration would also allow the use of multiple cutting profiles and cutting shapes of cutting edge 132 shown in FIG. 8 and FIG. 9, such as but not limited to: a toothed or swept cutting edge 132 of the blade element 130 with the effect of reducing the peak force required to perform the cut in the ostomy wafer 180 (FIG. 19).

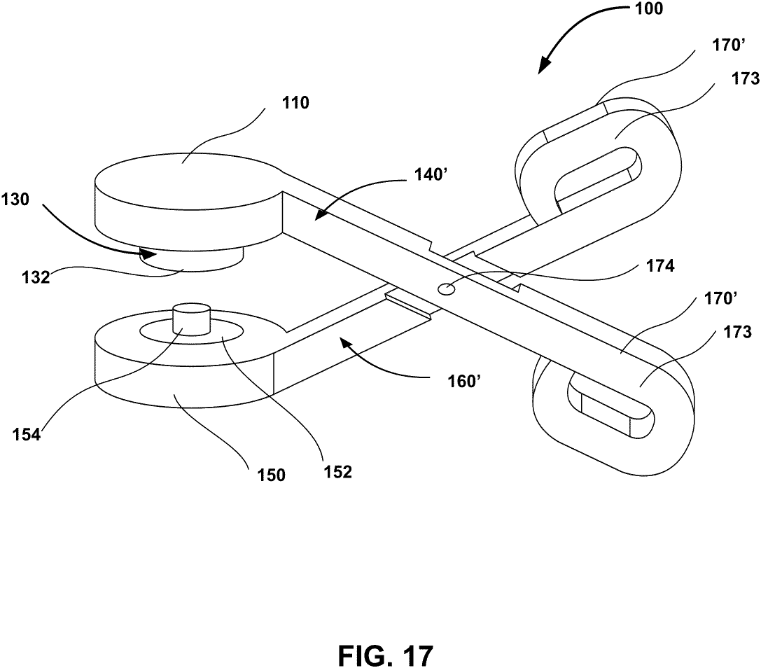

In an embodiment as shown in FIG. 17, that depicts a perspective view of an ostomy wafer cutter 100‴ according to another embodiment of the present invention. The ostomy wafer cutter 100‴ is similar to the ostomy wafer cutter 100 shown in FIG. 1 except for modifications associated with the handle portion, upper arm and the lower arm. A handle portion 170′ is positioned on both upper arm 140′ and lower arm 160′ for manipulating the position of upper arm 140′ relative to the lower arm 160′. As seen in FIG. 17, the handle portion 170′ comprises a pair of scissor handles 173. The scissor handles 173 are pivoted to each other in usual manner by a hinge element 174 i.e. screw.

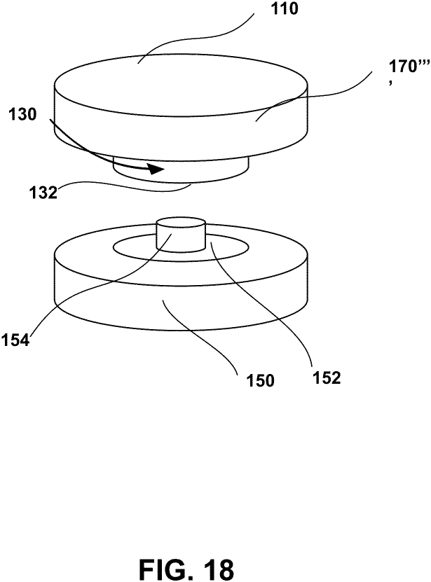

In an embodiment as shown in FIG. 18, that depicts a perspective view of an ostomy wafer cutter 100”” according to another embodiment of the present invention. The ostomy wafer cutter 100”” has a hinge-less design and the ostomy wafer cutter 100”” does not comprise any upper arm, lower arm or a hinge element connected to upper arm and lower arm. Instead, the handle portion 170”” is positioned on the upper element 110. The central pin 154 is inserted in a hole 182 (FIG. 19) of the ostomy wafer 180 (FIG. 19). This configuration allows a user to perform a variety of movements of the upper arm 110 to cut the ostomy wafer 180 (FIG. 19) placed between the upper arm 110 and the lower arm 150. The variety of movements of the upper arm 110 could include: rotational movement, longitudinal movement, and a combination of rotational movement and longitudinal movement. This flexibility of use of the ostomy wafer cutter 100⁗ enables a user to perform cutting of the ostomy wafer 180 according to their customary requirement.

The various components, parts of the various embodiments of the ostomy wafer cutter of the present invention are similar and interchangeable. It is obvious to the one skilled in the art that the various components, parts of the ostomy wafer cutter of one embodiment of the present invention could be considered for other embodiments with little or no variation.

It should be understood according to the preceding description of the present invention that the same is susceptible to changes, modifications and adaptations, and that the said changes, modifications and adaptations fall within scope of the appended claims.

What is claimed is:

1. An ostomy wafer cutter (100) for enlarging a hole (182) of an ostomy wafer (180) comprising:
an upper element (110) comprising a locking means (120) to removably lock a blade element (130), wherein the blade element (130) comprises a cutting edge (132);
an upper arm (140) connected to an upper element (110);
a lower element (150) comprising a platform (152) and a central pin (154) arranged in the platform (152) for insertion in the hole (182) of the ostomy wafer (180);
a lower arm (160) connected to a lower element (150); and
a handle portion (170) positioned on atleast one of: upper arm (140) and/or lower arm (160), wherein a user may grasp and move the handle portion (170) in a cutting position to bring the upper element (110) in close contact with the lower element (150); causing the cutting edge (132) to cut the ostomy wafer (180) and thereby enlarging a hole (182) of the ostomy wafer (180).

2. The ostomy wafer cutter (100) according to claim 1, wherein the lower arm (160) is rotatably connected to the upper arm (140) by a hinge element (190).

3. The ostomy wafer cutter (100) according to claim 1, wherein the upper arm (140) is detachably connected to an upper element (110) and the lower arm (160) is detachably connected to the lower element (150) respectively.

4. The ostomy wafer cutter (100) according to claim 1, wherein the blade element (130) comprises a biasing spring (136) to restore the upper element (110) relative to the lower element (150) in the initial position.

5. The ostomy wafer cutter (100) according to claim 1, wherein a spring (165) is positioned between the upper arm (140) and the lower arm (160) to restore the upper arm (140) relative to the lower arm (160) in the initial position.

6. The ostomy wafer cutter (100) according to claim 1, wherein the locking means (120) comprises atleast one of: a twist lock arrangement, luer locks, screw arrangements, magnetic couplings, mechanical locks, friction locks, snap fit couplings, keys and keyways.

7. The ostomy wafer cutter (100) according to claim 1, wherein the platform (152) is removably connected to the lower element (150).

8. The ostomy wafer cutter (100) according to claim 1, wherein the platform (152) has a cutting edge (156).

9. The ostomy wafer cutter (100) according to claim 1, wherein the platform (152) comprises a circumferential slot (157).

10. The ostomy wafer cutter (100) according to claim 1, wherein the handle portion (170) comprises a pair of scissors handles (173).

11. The ostomy wafer cutter (100) according to claim 1, wherein the handle portion (170) comprises a stapler handles.
